(19) United States
(12) Patent Application Publication
(10) Pub. No.: US 2012/0076775 A1
(43) Pub. Date: Mar. 29, 2012

(54) Combination of HGF Inhibitor and EGF Inhibitor to Treat Cancer

(76) Inventors: John Laterra, Baltimore, MD (US); Bachchu Lal, Pikesville, MD (US); Kyung Jin Kim, Cupertino, CA (US)

(21) Appl. No.: 12/963,595

(22) Filed: Dec. 8, 2010

Related U.S. Application Data
(63) Continuation of application No. 12/421,556, filed on Apr. 9, 2009, now abandoned.
(60) Provisional application No. 61/044,440, filed on Apr. 11, 2008.

(51) Int. Cl. A61K 39/395; A61K 31/5377; A61K 31/517; A61P 35/00

(52) U.S. Cl. 424/133.1; 424/142.1; 514/234.5; 514/266.4

(57) ABSTRACT

The present invention is directed toward a method of treating cancer by administering to a patient an inhibitor of Hepatocyte Growth Factor and an inhibitor of, e.g., Epidermal Growth Factor.

### CROSS-REFERENCE TO RELATED APPLICATIONS

This application claims the benefit under 35 U.S.C. §119(e) of U.S. Patent Application No. 61/044,440 filed Apr. 11, 2008, which is herewith incorporated in its entirety for all purposes.

### STATEMENT OF GOVERNMENT INTEREST

The invention described in this application was made in part with funding by Grants 5R44 CA101283-03 and RO1 CA129192 from the National Institutes of Health. The US Government has certain rights in this invention.

### REFERENCE TO A “SEQUENCE LISTING,” A TABLE, OR A COMPUTER PROGRAM LISTING SUBMITTED IN COMPUTER READABLE FORMAT

The Sequence Listing written in file 022382-000510US_SEQ.txt is 17,787 bytes, and was created on Apr. 9, 2009, for the application filed herewith, Laterra et al. “COMBINATION OF HGF INHIBITOR AND EGF INHIBITOR TO TREAT CANCER.” The information contained in this file is hereby incorporated by reference.

### FIELD OF THE INVENTION

The present invention relates generally to the treatment of cancer, and more particularly, for example, to treatment of cancer with an agent that inhibits hepatocyte growth factor together with an agent that blocks another cellular signaling pathway.

### BACKGROUND OF THE INVENTION

Human Hepatocyte Growth Factor (HGF) is a multifunctional heterodimeric polypeptide produced by mesenchymal cells. HGF has been shown to stimulate angiogenesis, morphogenesis and motogenesis, as well as the growth and scattering of various cell types (Bussolino et al., J. Cell. Biol. 119: 629, 1992; Zarnegar and Michalopoulos, J. Cell. Biol. 129:1177, 1995; Matsumoto et al., Ciba. Found. Symp. 212:198, 1997; Birchmeier and Gherardi, Trends Cell. Biol. 8:404, 1998; Xin et al. Am. J. Pathol. 158:1111, 2001). The pleiotropic activities of HGF are mediated through its receptor, a transmembrane tyrosine kinase encoded by the proto-oncogene cMet. In addition to regulating a variety of normal cellular functions, HGF and its receptor c-Met have been shown to be involved in the initiation, invasion and metastasis of tumors (Jeffers et al., J. Mol. Med. 74:505, 1996; Comoglio and Trusolino, J. Clin. Invest. 109:857, 2002). HGF/cMet are coexpressed, often over-expressed, on various human solid tumors including tumors derived from lung, colon, rectum, stomach, kidney, ovary, skin, multiple myeloma and thyroid tissue (Prat et al., Int. J. Cancer 49:323, 1991; Chan et al., Oncogene 2:593, 1988; Weidner et al., Am. J. Respir. Cell. Mol. Biol. 8:229, 1993; Derksen et al., Blood 99:1405, 2002). HGF acts as an autocrine (Rong et al., Proc. Natl. Acad. Sci. USA 91:4731, 1994; Koochekpour et al., Cancer Res. 57:5391, 1997) and paracrine growth factor (Weidner et al., Am. J. Respir. Cell. Mol. Biol. 8:229, 1993) and anti-apoptotic regulator (Gao et al., J. Biol. Chem. 276:47257, 2001) for these tumors.

HGF is a 102 kDa protein with sequence and structural similarity to plasminogen and other enzymes of blood coagulation (Nakamura et al., Nature 342:440, 1989; Weidner et al., Am. J. Respir. Cell. Mol. Biol. 8:229, 1993, each of which is incorporated herein by reference). Human HGF is synthesized as a 728 amino acid precursor (preproHGF), which undergoes intracellular cleavage to an inactive, single chain form (proHGF) (Nakamura et al., Nature 342:440, 1989; Rosen et al., J. Cell. Biol. 127:1783, 1994). Upon extracellular secretion, proHGF is cleaved to yield the biologically active disulfide-linked heterodimeric molecule composed of an α-subunit and β-subunit (Nakamura et al., Nature 342:440, 1989; Naldini et al., EMBO J. 11:4825, 1992). The α-subunit contains 440 residues (69 kDa with glycosylation), consisting of the N-terminal hairpin domain and four kringle domains. The (3-subunit contains 234 residues (34 kDa) and has a serine protease-like domain, which lacks proteolytic activity. Cleavage of HGF is required for receptor activation, but not for receptor binding (Hartmann et al., Proc. Natl. Acad. Sci. USA 89:11574, 1992; Lokker et al., J. Biol. Chem. 268:17145, 1992). HGF contains 4 putative N-glycosylation sites, 1 in the α-subunit and 3 in the β-subunit. HGF has 2 unique cell specific binding sites: a high affinity (Kd=2×10⁻¹⁰ M) binding site for the cMet receptor and a low affinity (Kd=10⁻⁹ M) binding site for heparin sulfate proteoglycans (HSPG), which are present on the cell surface and extracellular matrix (Naldini et al., Oncogene 6:501, 1991; Bardelli et al., J. Biotechnol. 37:109, 1994; Sakata et al., J. Biol. Chem., 272:9457, 1997).

cMet is a member of the class IV protein tyrosine kinase receptor family. The full length cMet gene was cloned and identified as the cMet proto-oncogene (Cooper et al., Nature 311:29, 1984; Park et al., Proc. Natl. Acad. Sci. USA 84:6379, 1987). The cMet receptor is initially synthesized as a single chain, partially glycosylated precursor, p170^{(MET)} (Park et al., Proc. Natl. Acad. Sci. USA 84:6379, 1987; Giordano et al., Nature 339:155, 1989; Giordano et al., Oncogene, 4:1383, 1989; Bardelli et al., J. Biotechnol., 37:109, 1994). Upon further glycosylation, the protein is proteolytically cleaved into a heterodimeric 190 kDa mature protein (1385 amino acids), consisting of the 50 kDa α-subunit (residues 1-307) and the 145 kDa β-subunit. The cytoplasmic tyrosine kinase domain of the β-subunit is involved in signal transduction.

Several different approaches have been investigated to obtain HGF inhibitors, i.e. antagonists. Such inhibitors include truncated HGF proteins such as NK1 (N terminal domain plus kringle domain 1; Lokker et al., J. Biol. Chem. 268:17145, 1993); NK2 (N terminal domain plus kringle domains 1 and 2; Chan et al., Science 254:1382, 1991); and NK4 (N-terminal domain plus four kringle domains), which was shown to partially inhibit the primary growth and metastasis of murine lung tumor LLC in a nude mouse model (Kuba et al., Cancer Res. 60:6737, 2000)

As another approach, Dodge (Master's Thesis, San Francisco State University, 1998) generated antagonist anti-cMet monoclonal antibodies (mAbs). One mAb, 5D5, exhibited strong antagonistic activity in ELISA, but induced a proliferative response of cMet-expressing BAF-3 cells, presumably due to dimerization of the membrane receptors. For this reason, a single-domain form of the anti-cMet mAb 5D5 has been developed as an antagonist (Nguyen et al., Cancer Gene Ther. 10:840, 2003).

Cao et al., Proc. Natl. Acad. Sci. USA 98:7443, 2001, reported that the administration of a cocktail of three anti-HGF mAbs, which were selected based upon their ability to inhibit the scattering activity of HGF in vitro, were able to inhibit the growth of human tumors in the xenograft nude mouse model.

More recently, several neutralizing (inhibitory) anti-HGF mAbs have been reported including L2G7 (Kim et al., Clin Cancer Res 12:1292, 2006 and U.S. Pat. No. 7,220,410), HuL2G7 (WO 07115049 A2), the human mAbs described in WO 2005/017107 A2, and the HGF binding proteins described in WO 07143090 A2 or WO 07143098 A2. It has also been reported that the anti-HGF mAb L2G7, when administered systemically, can strongly inhibit growth or even induce regression of orthotopic (intracranial) glioma xenografts and prolong animal survival (Kim et al., op. cit. and WO 06130773 A2).

Epidermal growth factor (EGF) is a widely distributed growth factor that in cancer, can stimulate cancer-cell proliferation, block apoptosis, activate invasion and metastasis, and stimulate angiogenesis (Citri et al., Nat. Rev. Mol. Cell. Biol. 7:505, 2006; Hynes et al., Nat. Rev. Cancer 5:341, 2005). The EGF receptor (EGFR or ErbB) is a transmembrane, tyrosine kinase receptor that belongs to a family of four related receptors. The majority of human epithelial cancers are marked by functional activation of growth factors and receptors of this family (Ciardiello et al., New Eng. J. Med. 358: 1160, 2008) so that EGF and EGFR are natural targets for cancer therapy. Activation of EGFR is commonly associated with mutations, for example of exons 19 and 21 in some lung cancers, or deletion of exons 2-7 to form EGF receptor variant III (EGFRvIII) in many gliomas (Rosell et al., Clin. Cancer Res. 12:7222, 2006; Ji et al., Proc. Natl. Acad. Sci. USA 103:7817, 2006).

Four inhibitors of the EGF/EGFR pathway have been approved for marketing as drugs: Erbitux® (cetuximab, a chimeric anti-EGFR mAb Anatomical Therapeutic Chemical (ATC) code L01XC06, commercially available from Imclone/Bristol Myers Squibb) for colon cancer and squamous-cell cancer of the head and neck cancer; Vectibix® (panitumumab, a human anti-EGFR mAb ATC code L01XC08) for colon cancer, commercially available from Amgen; Tarceva® (erlotinib, N-(3-ethynylphenyl)-6,7-bis(2-methoxyethoxy)-4-quinazolinamine, commercially available from Genentech) and Iressa® (gefitinib 4-Quinazolinamine, N-(3-chloro-4-fluorophenyl)-7-methoxy-6-[3-4-morpholin) propoxy], commercially available from AstraZeneca), both small molecule inhibitors of the tyrosine kinase activity of EGFR, with Tarceva for the treatment of non-small-cell lung cancer and pancreatic cancer and Iressa for the treatment of non-small-cell lung cancer in special circumstances. However, cancer cells can rapidly switch their dependence from EGFR to cMet (RTK Switching; Stommel et al., Science 318:287, 2007), and EGFR-dependent tumors can develop resistance to the EGFR inhibitors erlotinib and geftinib inhibitors by amplification of cMet (Bean et al., Proc. Natl. Acad. Sci. USA 104:20932, 2007).

### SUMMARY OF THE INVENTION

The invention provides a method of treating cancer by administering to a patient in need of such treatment a first agent that inhibits Hepatocyte Growth Factor (HGF) in combination with a second agent that inhibits a signaling pathway other than the one stimulated by HGF (the HGF/cMet pathway). In a preferred embodiment, the first agent is a monoclonal antibody (mAb) that binds to and neutralizes HGF. Chimeric, human and humanized anti-HGF mAbs are especially preferred, particularly humanized L2G7. In some embodiments, the second agent is an inhibitor of epidermal growth factor (EGF), for example a mAb that binds to the EGF receptor, thereby inhibiting binding of EGF, such as cetuximab or panitumumab; or alternatively a small molecule inhibitor of the EGF pathway such as erlotinib or gefitinib. The method is especially preferred for treating lung, colon, head and neck cancer and brain tumors such as glioma.

### BRIEF DESCRIPTION OF THE DRAWINGS

FIG. 1. Graph of tumor growth vs days after tumor implantation of GB-d1 gallbladder tumor xenografts in mice treated with PBS, anti-HGF mAb HuL2G7 (also known as TAK-701), anti-EGFR mAb M225 or a combination of HuL2G7 and M225.

FIG. 2. Graph of tumor growth vs days after tumor implantation of U87EGFRvIII xenografts in mice treated with control mAb 5G8, anti-HGF mAb HuL2G7 (also known as TAK-701), EGFR antagonist erlotinib, or L2G7 in combination with erlotinib. Arrows show days on which mAbs were administered.

FIG. 3. Graph of survival of mice with U87EGFRvIII intracranial xenografts treated with control mAb 5G8, anti-HGF mAb L2G7, 5G8 plus erlotinib, or L2G7 plus erlotinib. The arrows delineate the period of treatment.

FIGS. 4A and 4B. Amino acid sequences of the entire HuL2G7 heavy chain (A) (SEQ ID NO:1) and light chain (B) (SEQ ID NO:2). The first amino acids of the mature heavy and light chain variable regions (i.e., after cleavage of the signal sequences) are double underlined and labeled with the number 1; these amino acids are therefore the first amino acids of the light and heavy chains of the actual HuL2G7 mAb. In the heavy chain, the first amino acids of the CH1, hinge, CH2 and CH3 regions are underlined, and in the light chain, the first amino acid of the C_{κ} region is underlined.

FIGS. 5A and 5B. Amino acid sequences of the light chain (A) (SEQ ID NO:3) and heavy chain (B) (SEQ ID NO:4) variable regions of the 2.12.1 human monoclonal antibody disclosed in WO 2005/017107 A2, therein designated respectively as SEQ ID NOS. 38 and 39. The first amino acids of the mature heavy and light variable regions (i.e., after cleavage of the signal sequences), and thus of the actual 2.12.1 mAb, are double underlined.

FIGS. 6A and 6B. Amino acid sequences of the light chain (A) (SEQ ID NO:5) and heavy chain (B) (SEQ ID NO:6) of Vectibix® (signal sequences not included). The C-terminal K of the heavy chain is cleaved during processing and not present to a significant extent in the final product.

FIGS. 7A and 7B. Amino acid sequences of the light chain variable region (A) (SEQ ID NO:7) and heavy chain variable region (B) (SEQ ID NO:8) of the M225 antibody. Signal sequences are included. The first amino acids of the mature variable heavy and light chain variable regions are double underlined.

### DETAILED DESCRIPTION OF THE PREFERRED EMBODIMENTS

The invention provides a method of treating cancer by administering to a patient in need of such treatment a first agent that inhibits the activity of Hepatocyte Growth Factor (HGF), i.e., an HGF antagonist or cMet antagonist, in combination with (i.e., together with) a second agent that inhibits a cellular signaling pathway other than the one stimulated by HGF (the HGF/cMet pathway). In many embodiments, the first agent and/or the second agent is a monoclonal antibody (mAb).

### 1. Antibodies

Antibodies are very large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions fold up together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-D space to form the actual antibody binding site which locks onto the target antigen. The position and length of the CDRs have been precisely defined. Kabat, E. et al., Sequences of Proteins of Immunological Interest, U.S. Department of Health and Human Services, 1983, 1987. The part of a variable region not contained in the CDRs is called the framework, which forms the environment for the CDRs.

A monoclonal antibody (mAb) is a single molecular species of antibody and therefore does not encompass polyclonal antibodies produced by injecting an animal (such as a rodent, rabbit or goat) with an antigen, and extracting serum from the animal. A humanized antibody is a genetically engineered monoclonal antibody in which the CDRs from a mouse antibody (“donor antibody”, which can also be rat, hamster or other similar species) are grafted onto a human antibody (“acceptor antibody”). Humanized antibodies can also be made with less than the complete CDRs from a mouse antibody (e.g., Pascalis et al., J. Immunol. 169:3076, 2002). Thus, a humanized antibody is an antibody having CDRs from a donor antibody and variable region frameworks and constant regions from human antibodies. The light and heavy chain acceptor frameworks may be from the same or different human antibodies and may each be a composite of two or more human antibody frameworks; or alternatively may be a consensus sequence of a set of human frameworks (e.g., a subgroup of human antibodies as defined in Kabat et al., op. cit.), i.e., a sequence having the most commonly occurring amino acid in the set at each position. In addition, in order to retain high binding affinity, at least one of two additional structural elements can be employed. See, U.S. Pat. Nos. 5,530,101 and 5,585,089, each of which is incorporated herein by reference, which provide detailed instructions for construction of humanized antibodies.

In the first structural element, the framework of the heavy chain variable region of the humanized antibody is chosen to have maximal sequence identity (between 65% and 95%) with the framework of the heavy chain variable region of the donor antibody, by suitably selecting the acceptor antibody from among the many known human antibodies. Sequence identity is determined when antibody sequences being compared are aligned according to the Kabat numbering convention. In the second structural element, in constructing the humanized antibody, selected amino acids in the framework of the human acceptor antibody (outside the CDRs) are replaced with corresponding amino acids from the donor antibody, in accordance with specified rules. Specifically, the amino acids to be replaced in the framework are chosen on the basis of their ability to interact with the CDRs. For example, the replaced amino acids can be adjacent to a CDR in the donor antibody sequence or within 4-6 angstroms of a CDR in the humanized antibody as measured in 3-dimensional space.

A chimeric antibody is an antibody in which the variable region of a mouse (or other rodent) antibody is combined with the constant region of a human antibody; their construction by means of genetic engineering is well-known. Such antibodies retain the binding specificity of the mouse antibody, while being about two-thirds human. The proportion of nonhuman sequence present in mouse, chimeric and humanized antibodies suggests that the immunogenicity of chimeric antibodies is intermediate between mouse and humanized antibodies. Other types of genetically engineered antibodies that may have reduced immunogenicity relative to mouse antibodies include human antibodies made using phage display methods (Dower et al., WO91/17271; McCafferty et al., WO92/001047; Winter, WO92/20791; and Winter, FEBS Lett. 23:92, 1998, each of which is incorporated herein by reference) or using transgenic animals (Lonberg et al., WO93/12227; Kucherlapati WO91/10741, each of which is incorporated herein by reference).

As used herein, the term “human-like” antibody refers to a mAb in which a substantial portion of the amino acid sequence of one or both chains (e.g., about 50% or more) originates from human immunoglobulin genes. Hence, human-like antibodies encompass but are not limited to chimeric, humanized and human antibodies. As used herein, a “reduced-immunogenicity” antibody is one expected to have significantly less immunogenicity than a mouse antibody when administered to human patients. Such antibodies encompass chimeric, humanized and human antibodies as well as antibodies made by replacing specific amino acids in mouse antibodies that may contribute to B- or T-cell epitopes, for example exposed residues (Padlan, Mol. Immunol. 28:489, 1991). As used herein, a “genetically engineered” antibody is one for which the genes have been constructed or put in an unnatural environment (e.g., human genes in a mouse or on a bacteriophage) with the help of recombinant DNA techniques, and would therefore, e.g., not encompass a mouse mAb made with conventional hybridoma technology.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 50% but preferably 75%, 90% or even 99% as measured in a competitive binding assay compared to a control lacking the competing antibody (see, e.g., Junghans et al., Cancer Res. 50:1495, 1990, which is incorporated herein by reference). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

### 2. Antibodies for Use in the Invention

A monoclonal antibody (mAb) that binds HGF (i.e., an anti-HGF mAb) is said to neutralize HGF, or be neutralizing, if the binding partially or completely inhibits one or more biological activities of HGF (i.e., when the mAb is used as a single agent). Among the biological properties of HGF that a neutralizing antibody may inhibit are the ability of HGF to bind to its cMet receptor, to cause the scattering of certain cell lines such as Madin-Darby canine kidney (MDCK) cells; to stimulate proliferation of (i.e., be mitogenic for) certain cells including hepatocytes, My 1 Lu mink lung epithelial cells, and various human tumor cells; or to stimulate angiogenesis, for example as measured by stimulation of human vascular endothelial cell (HUVEC) proliferation or tube formation or by induction of blood vessels when applied to the chick embryo chorioallantoic membrane (CAM). Antibodies for use in the invention preferably bind to human HGF, i.e., to the protein encoded by the GenBank sequence with Accession number D90334.

A neutralizing anti-HGF mAb is preferred for use as the first agent in the invention and, at a concentration of, e.g., 0.01, 0.1, 0.5, 1, 2, 5, 10, 20 or 50 μg/ml, inhibits a biological function of HGF (e.g., stimulation of proliferation or scattering) by about at least 50% but preferably 75%, more preferably by 90% or 95% or even 99%, and most preferably approximately 100% (essentially completely) as assayed by methods known in the art. Inhibition is considered complete if the level of activity is within the margin of error for a negative control lacking HGF. Typically, the extent of inhibition is measured when the amount of HGF used is just sufficient to fully stimulate the biological activity, or is 0.05, 0.1, 0.5, 1, 3 or 10 μg/ml. Preferably, at least 50%, 75%, 90%, or 95% or essentially complete inhibition is achieved when the molar ratio of antibody to HGF is 0.5×, 1×, 2×, 3×, 5× or 10×. Preferably, the mAb is neutralizing, i.e., inhibits the biological activity, when used as a single agent, but optionally 2 mAbs can be used together to give inhibition. Most preferably, the mAb neutralizes not just one but several of the biological activities listed above; for purposes herein, an anti-HGF mAb that used as a single agent neutralizes all the biological activities of HGF is called “fully neutralizing”, and such mAbs are most preferable. Anti-HGF mAbs for use in the invention are preferably specific for HGF, that is they do not bind, or only bind to a much lesser extent (e.g., Ka at least ten-fold less), proteins that are related to HGF such as fibroblast growth factor (FGF) and vascular endothelial growth factor (VEGF). Preferred antibodies lack agonistic activity toward HGF. That is, the antibodies block interaction of HGH with cMet without stimulating cells bearing HGF directly. Anti-HGF mAbs for use in the invention typically have a binding affinity (Kₐ) for HGF of at least 10⁷ M⁻¹ but preferably 10⁸ M⁻¹ or higher, and most preferably 10⁹ M⁻¹ or higher or even 10¹⁰ M⁻¹ or higher.

MAbs for use in the invention include antibodies in their natural tetrameric form (2 light chains and 2 heavy chains) and may be of any of the known isotypes IgG, IgA, IgM, IgD and IgE and their subtypes, i.e., human IgG1, IgG2, IgG3, IgG4 and mouse IgG1, IgG2a, IgG2b, and IgG3. The mAbs are also meant to include fragments of antibodies such as Fv, Fab and F(ab′)₂; bifunctional hybrid antibodies (e.g., Lanzavecchia et al., Eur. J. Immunol. 17:105, 1987), single-chain antibodies (Huston et al., Proc. Natl. Acad. Sci. USA 85:5879, 1988; Bird et al., Science 242:423, 1988); single-arm antibodies (Nguyen et al., Cancer Gene Ther. 10:840, 2003); and antibodies with altered constant regions (e.g., U.S. Pat. No. 5,624,821). The mAbs may be of animal (e.g., mouse, rat, hamster or chicken) origin, or they may be genetically engineered. Rodent mAbs are made by standard methods well-known in the art, comprising multiple immunization with HGF in appropriate adjuvant i.p., i.v., or into the footpad, followed by extraction of spleen or lymph node cells and fusion with a suitable immortalized cell line, and then selection for hybridomas that produce antibody binding to HGF, e.g., see under Examples. Chimeric and humanized mAbs, made by art-known methods mentioned supra, are preferred for use in the invention. Human antibodies made, e.g., by phage display or transgenic mice methods are also preferred (see e.g., Dower et al., McCafferty et al., Winter, Lonberg et al., Kucherlapati, supra). More generally, human-like, reduced immunogenicity and genetically engineered antibodies as defined herein are all preferred.

The neutralizing anti-HGF mAb L2G7 (which is produced by a hybridoma deposited at the American Type Culture Collection under ATCC Number PTA-5162 according to the Budapest treaty) as described in Kim et al., Clin Cancer Res 12:1292, 2006 and U.S. Pat. No. 7,220,410 and particularly its chimeric and humanized forms such as HuL2G7, as described in WO 07115049 A2, are especially preferred as the first agent in the invention. Neutralizing mAbs with the same or overlapping epitope as L2G7 and/or that compete with L2G7 for binding to HGF are also preferred. MAbs that are 90%, 95% or 99% identical to L2G7 in amino acid sequence, when aligned according to the Kabat numbering convention, at least in the CDRs, and maintain its functional properties, or which differ from it by a small number of functionally inconsequential amino acid substitutions (e.g., conservative substitutions), deletions, or insertions can also be used in the invention.

Also preferred for use as the first agent in the invention are the anti-HGF mAbs described in WO 2005/017107 A2, whether explicitly by name or sequence or implicitly by description or relation to explicitly described mAbs. Especially preferred mAbs are those produced by the hybridomas designated therein as 1.24.1, 1.29.1, 1.60.1, 1.61.3, 1.74.3, 1.75.1, 2.4.4, 2.12.1, 2.40.1 and 3.10.1, and respectively defined by their heavy and light chain variable region sequences provided by SEQ ID NO's 24-43, with 2.12.1 being most preferred; mAbs possessing the same respective CDRs as any of these listed mAbs; mAbs having light and heavy chain variable regions that are at least 90%, 95% or 99% identical to the respective variable regions of these listed mAbs or differing from them only by inconsequential amino acid substitutions, deletion or insertions; mAbs binding to the same epitope of HGF as any of these listed mAbs, and all mAbs encompassed by claims 1 through 94 therein.

Alternatively, any of the HGF binding proteins described in WO07143090A2 or WO07143098A2 may be used as the first agent in the invention.

Native mAbs for use in the invention may be produced from their hybridomas. Genetically engineered mAbs, e.g., chimeric or humanized mAbs, may be expressed by a variety of art-known methods. For example, genes encoding their light and heavy chain V regions may be synthesized from overlapping oligonucleotides and inserted together with available C regions into expression vectors (e.g., commercially available from Invitrogen) that provide the necessary regulatory regions, e.g., promoters, enhancers, poly A sites, etc. Use of the CMV promoter-enhancer is preferred. The expression vectors may then be transfected using various well-known methods such as lipofection or electroporation into a variety of mammalian cell lines such as CHO or non-producing myelomas including Sp2/0 and NS0, and cells expressing the antibodies selected by appropriate antibiotic selection. See, e.g., U.S. Pat. No. 5,530,101. Larger amounts of antibody may be produced by growing the cells in commercially available bioreactors.

Once expressed, the mAbs for use in the invention may be purified according to standard procedures of the art such as microfiltration, ultrafiltration, protein A or G affinity chromatography, size exclusion chromatography, anion exchange chromatography, cation exchange chromatography and/or other forms of affinity chromatography based on organic dyes or the like. Substantially pure antibodies of at least about 90 or 95% homogeneity are preferred, and 98% or 99% or more homogeneity most preferred, for pharmaceutical uses. The mAbs are typically provided in a pharmaceutical formulation, i.e., in a physiologically acceptable carrier, optionally with excipients or stabilizers. Acceptable carriers, excipients or stabilizers are nontoxic to recipients at the dosages and concentrations employed, and include buffers such as phosphate, citrate, or acetate at a pH typically of 5.0 to 8.0, most often 6.0 to 7.0; salts such as sodium chloride, potassium chloride, etc. to make isotonic; antioxidants, preservatives, low molecular weight polypeptides, proteins, hydrophilic polymers such as polysorbate 80, amino acids, carbohydrates, chelating agents, sugars, and other standard ingredients known to those skilled in the art (Remington's Pharmaceutical Science 16^{th} edition, Osol, A. Ed. 1980). The mAb is typically present at a concentration of 1-100 mg/ml, e.g., 10 mg/ml.

### 3. Other Agents for Use in the Invention

Besides anti-HGF mAbs, the first agent for use in the invention may be any other agent that inhibits HGF, i.e., inhibits its biological activity, and may therefore be called an HGF antagonist. Examples are soluble forms of cMet (e.g., see Michieli et al., Cancer Cell 6:61, 2004) and a cocktail of several anti-HGF mAbs (Cao et al., Proc. Natl. Acad. Sci. USA 98:7443, 2001). As used herein the term “agent that inhibits HGF” or “HGF inhibitor” also includes an agent that interacts with the cMet receptor of HGF so as to inhibit HGF signaling through cMet; such an agent may also be called a cMet inhibitor or antagonist. However, as used herein, inhibitors or antagonists of HGF or cMet or the HGF/cMet pathway are not meant to include agents that inhibit signaling events, such as activation of MAP kinase, that occur after (i.e., downstream) of the HGF-cMet interaction and activation of cMet, and which the HGF/cMet pathway shares with other ligand/receptor systems. A cMet antagonist may function by binding to cMet and competitively blocking binding of HGF or activation by HGF. Exemplary agents include truncated HGF proteins such as NK1, NK2, and NK4 (supra) and anti-cMet mAbs. A preferred example is an anti-cMet antibody that has been genetically engineered to have only one “arm”, i.e. binding domain, such as OA-5D5 (Martens et al., Clin. Cancer Res. 12:6144, 2006). Such agents may also be small molecule inhibitors of the tyrosine kinase activity of cMet including SU5416 (Wang et al., J Hepatology 41:267, 2004), and ARQ 197 being developed by ArQule, Inc. (Abstract Number 3525 at the 2007 Annual Meeting of the American Society of Clinical Oncology), which may be administered orally.

The second agent for use in the invention is any inhibitor of a cellular signaling pathway other than the HGF/cMet pathway. Such an agent may bind to the ligand stimulating the pathway or to its receptor or to a downstream signaling molecule. The agent may be a protein such as a mAb, preferably a chimeric, humanized or human mAb, which binds to the ligand or receptor, or may be a small molecule (i.e., a compound having relatively low molecular weight, most often less than 500 or 600 or 1000 kDa). Proteins are typically administered parenterally, e.g. intravenously, whereas small molecules may be administered parenterally or orally. The ligand is often a cytokine or growth factor, whereas the receptor is often a tyrosine kinase, so that tyrosine kinase inhibitors are preferred as a second agent in the invention. For example, the second agent may be an agent that inhibits EGF, preferably human EGF, i.e., inhibits its biological activity. An “agent that inhibits EGF” or “EGF inhibitor” includes an agent that interacts with the EGFR, preferably the human EGFR, so as to inhibit EGF signaling through EGFR; such an agent may also be called an EGFR inhibitor or antagonist. An EGFR antagonist may function by binding to EGFR and competitively blocking binding of EGF or activation by EGF, for example the anti-EGFR mAbs cetuximab and panitumumab, or by inhibiting the tyrosine kinase activity of EGFR, for example erlotinib and gefitinib. EGF and EGFR are well known human proteins for which sequences are available from UniProtKB/Swiss-Prot and similar databases. Insofar as a protein has more than one known form in a species due to natural allelic variation between individuals, an inhibitor can bind to and inhibit any, or all, of such known allelic forms, and preferably binds to and inhibits the wild type, most common or first published allelic form. Exemplary sequences for EGF and EGFR are assigned UniProtKB/Swiss-Prot accession numbers P01133 and P00533 respectively. More generally, downstream signaling pathways that may be inhibited by the second agent in the invention include the RAS-RAF-MEK-APK pathway and the PI3K-AKT pathway. Many other signaling pathways and their inhibitors are well known to those skilled in the art of cellular biology.

### 4. Treatment Methods

The invention provides methods of treatment in which the indicated first and second agents are administered to patients having a cancer (therapeutic treatment) or at risk of occurrence or recurrence of cancer (prophylactic treatment). The term “patient” includes human patients; veterinary patients, such as cats, dogs and horses; farm animals, such as cattle, sheep, and pigs; and laboratory animals used for testing purposes, such as mice and rats. The methods are particularly amenable to treatment of human patients. In some methods, the patient has a tumor including cells with a mutation in an EGFR receptor, such as a deletion of exons 2-7. Optionally, a tumor biopsy can be tested for such mutations at the DNA or protein level before treatment. The mAb or other agent used in methods of treating human patients binds to the respective human protein. A mAb or other agent to a human protein can also be used in other species in which the species homolog has antigenic crossreactivity with the human protein. In species lacking such crossreactivity, an antibody or other agent is used with appropriate specificity for the species homolog present in that species. However, in xenograft experiments in laboratory animals, a mAb or other agent with specificity for the human protein expressed by the xenograft is generally used.

A mAb or other protein used as a first or second agent in the methods of the invention can be administered to a patient by any suitable route, especially parentally by intravenous (IV) infusion or bolus injection, intramuscularly or subcutaneously or intraperitoneally. IV infusion can be given over as little as 15 minutes, but more often for 30 minutes, 60 minutes, 90 minutes or even 2 or 3 hours. The agent can also be injected directly into the site of disease (e.g., the tumor itself; or the brain or its surrounding membranes or cerebrospinal fluid in the case of a brain tumor) or encapsulated into carrying agents such as liposomes. However, when treating brain tumors (i.e., a tumor existing within the brain of a patient), systemic administration of the mAb, e.g., by IV infusion, is possible and even preferred (see WO 06130773 A2). The dose given to a patient having a cancer is sufficient to alleviate or at least partially arrest the disease being treated (“therapeutically effective dose”) and is sometimes 0.1 to 5 mg/kg body weight, for example 1, 2, 3, 4, 5 or 6 mg/kg, but may be as high as 10 mg/kg or even 15 or 20 or 30 mg/kg. A fixed unit dose may also be given, for example, 50, 100, 200, 500 or 1000 mg, or the dose may be based on the patient's surface area, e.g., 100 mg/m². Usually between 1 and 8 doses, (e.g., 1, 2, 3, 4, 5, 6, 7 or 8) are administered to treat cancer, but 10, 12, 20 or more doses may be given. The agent can be administered daily, biweekly, weekly, every other week, monthly or at some other interval, depending, e.g. on its half-life, for 1 week, 2 weeks, 4 weeks, 8 weeks, 3-6 months or longer, or until the disease progresses. Repeated courses of treatment are also possible, as is chronic administration.

When a small molecule is used as the first or second agent, it is typically administered more often, preferably once a day, but 2, 3, 4 or more times per day is also possible, as is every two days, weekly or at some other interval. Small molecule drugs are often taken orally but parenteral administration is also possible, e.g., by IV infusion or bolus injection or subcutaneously or intramuscularly. Doses of small molecule drugs are typically 10 to 1000 mg, with 100, 150, 200 or 250 mg very typical, with the optimal dose established in clinical trials. For either a protein or small molecule drug, a regime of a dosage and intervals of administration that alleviates or at least partially arrests the symptoms of a disease (biochemical, histologic and/or clinical), including its complications and intermediate pathological phenotypes in development of the disease is referred to as a therapeutically effective regime.

When a first agent (an HGF inhibitor) is used in combination with a second agent (e.g., an EGF inhibitor), the combination may take place over any convenient timeframe. For example, each agent may be administered to a patient on the same day, and the agents may even be administered in the same intravenous infusion. However, the agents may also be administered on alternating days or alternating weeks, fortnights or months, and so on. In some methods, the respective agents are administered with sufficient proximity in time that the agents are simultaneously present (e.g., in the serum) at detectable levels in the patient being treated. In some methods, an entire course of treatment of one agent consisting of a number of doses over a time period (see above) is followed by a course of treatment of the other agent also consisting of a number of doses. In some methods, treatment with the agent administered second is begun if the patient has resistance or develops resistance to the agent administered initially. The patient may receive only a single course of treatment with each agent or multiple courses with one or both agents. Frequently, a recovery period of 1, 2 or several days or weeks is allowed between administration of the two agents if this is beneficial to the patient in the judgment of the attending physician. When a suitable treatment regiment has already been established for one of the agents, that regimen is preferably used when the agent in used in combination with the other. For example, Tarceva® (erlotinib) is taken as a 100 mg or 150 mg pill once a day, and Iressa® (gefitinib) is taken as 250 mg tablet daily. Erbitux® (cetuximab) is administered as an IV infusion in an initial dose of 400 mg/m² followed by weekly 250 mg/m² doses, and Vectibix® (panitumumab) is administered as an IV infusion of 6 mg/kg every 2 weeks. Typically, these agents are administered until the disease progresses

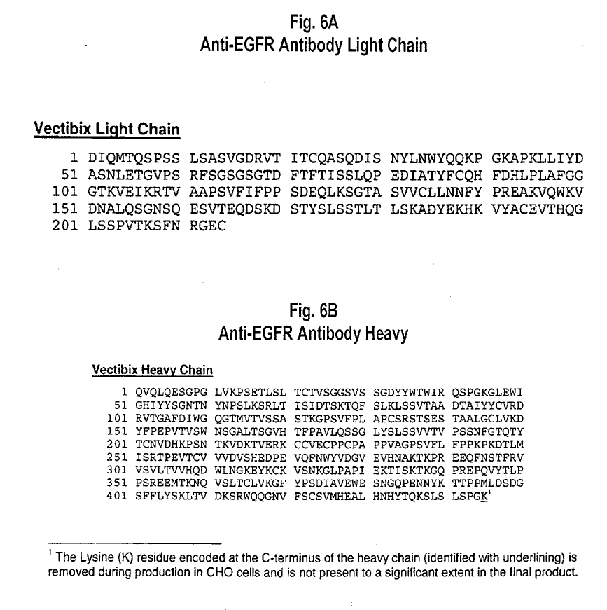

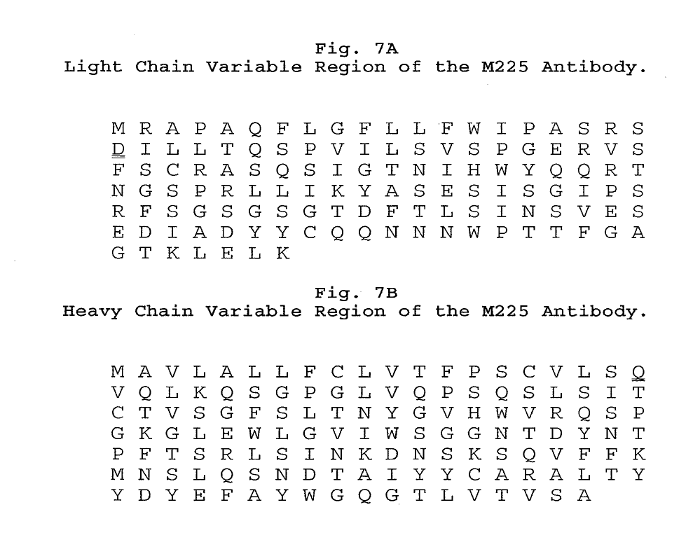

Sequences from the heavy and light chain variable region of several human anti-EGFR antibodies that can be used in the present methods are disclosed in U.S. Pat. No. 6,235,883 (incorporated by reference) The full length sequences of Vectibix (not including signal sequences) are reproduced in FIGS. 6A and 6B (see Amgen submission for patent term extension of '833 patent)). Erbitux is a chimeric form (human IgG1 kappa) of a mouse 225 antibody described in U.S. Pat. No. 4,943,533. Amino acid sequences of the light and heavy chain variable regions of this antibody are described in U.S. Pat. No. 7,060,808 (incorporated by reference) and reproduced in FIGS. 7A and 7B. The '808 patent also describes a humanized form of the 225 antibody. This humanized antibody can also be used in the present methods.

Optionally, an HGF and an EGF inhibitor can be combined in a kit, for example, as separate vials in the same package, or holder. The kit can contain instructions for performing any of the methods described herein. Some combinations of EGF and HGF inhibitors (for example, two antibodies, can also be mixed in the same composition. Such composition and kits can be formed either by a manufacturer or by a health care provider.

The methods of the invention can also be used in prophylaxis of a patient at risk of cancer. Such patients include those having genetic susceptibility to cancer, patients who have undergone exposure to carcinogenic agents, such as radiation or toxins, and patients who have undergone previous treatment for cancer and are at risk of recurrence. A prophylactic dosage is an amount sufficient to eliminate or reduce the risk, lessen the severity, or delay the outset of the disease, including biochemical, histologic and/or clinical symptoms of the disease, its complications and intermediate pathological phenotypes presenting during development of the disease. Administration of a pharmaceutical composition in an amount and at intervals effective to effect one or more of these objects is referred to as a prophylactically effective regime. The dosages and regimens disclosed above for therapeutic treatment can also be used for prophylactic treatment.

Types of cancer especially susceptible to treatment using the methods of the invention include solid tumors known or suspected to require angiogenesis or to be associated with elevated levels of HGF or cMet (which can be measured at the mRNA or protein level relative to noncancerous tissue of the same type, optionally from the same patient), for example ovarian cancer, breast cancer, lung cancer (small cell or non-small cell), colon cancer, prostate cancer, pancreatic cancer, bladder cancer, cervical cancer, renal cancer, gastric cancer, liver cancer, head and neck tumors, mesothelioma, melanoma, and sarcomas, and brain tumors. Treatment can also be administered to patients having leukemias or lymphomas. The methods of the invention are particularly suitable for treatment of brain tumors including meningiomas; gliomas including ependymomas, oligodendrogliomas, and all types of astrocytomas (low grade, anaplastic, and glioblastoma multiforme or simply glioblastoma); medullablastomas, gangliogliomas, schwannomas, chordomas; and brain tumors primarily of children including primitive neuroectodermal tumors. Both primary brain tumors (i.e., arising in the brain) and secondary or metastatic brain tumors can be treated by the methods of the invention. When the second agent is an EGF inhibitor, tumors known to be susceptible to one or more of the approved EGF inhibitor drugs are especially preferred, e.g., lung, colon, head and neck, and brain cancer. Tumor types or individual tumors in which the EGFR is over-active, typically because of mutation (e.g., EGFRvIII) or amplification, are most preferred as the target of treatment.

Because of the severity of cancer, several drugs to treat the disease are often given in combination. Hence, in a preferred embodiment of the present invention, the first agent (an HGF inhibitor) and the second agent (e.g., an EGF inhibitor) are administered together with additional anti-cancer drugs. The first agent and second agent can be administered before, during or after the other anti-cancer drugs. For example, the first and second agents may be administered together with any one or more of the chemotherapeutic drugs known to those of skill in the art of oncology, for example alkylating agents such as carmustine, chlorambucil, cisplatin, carboplatin, oxaliplatin, procarbazine, and cyclophosphamide; antimetabolites such as fluorouracil, floxuridine, fludarabine, gemcitabine, methotrexate and hydroxyurea; natural products including plant alkaloids and antibiotics such as bleomycin, doxorubicin, daunorubicin, idarubicin, etoposide, mitomycin, mitoxantrone, vinblastine, vincristine, and Taxol (paclitaxel) or related compounds such as Taxotere®; the topoisomerase 1 inhibitor irinotecan; agents specifically approved for brain tumors including temozolomide and Gliadel® wafer containing carmustine; and inhibitors of tyrosine kinases such as Gleevec® and Sutent® (sunitinib malate); and all approved and experimental anti-cancer agents listed in WO 2005/017107 A2 (which is herein incorporated by reference). The first and second agents can be administered in combination with 1, 2, 3 or more of these other agents used in a standard chemotherapeutic regimen. Normally, the other agents are those already known to be effective for the particular type of cancer being treated. Moreover, the first and second agents can be administered together with any form of radiation therapy including external beam radiation, intensity modulated radiation therapy (IMRT) and any form of radiosurgery including Gamma Knife, Cyberknife, Linac, and interstitial radiation (e.g. implanted radioactive seeds, GliaSite balloon), and/or with surgery. Combination with radiation therapy can be especially appropriate for head and neck cancer and brain tumors. Other agents with which the first and second agents can be administered include biologics such as monoclonal antibodies, including Herceptin™ against the HER2 antigen and Avastin™ against VEGF.

The progression-free survival or overall survival time of patients with cancer (e.g., ovarian, prostate, breast, lung, colon, pancreas, kidney, head and neck, and brain, especially when relapsed or refractory) treated according to the method of the invention with the first and second agents may increase by at least 10%, 20%, 30% or 40% but preferably 50%, 60% to 70% or even 80%, 90%, 100% or longer, compared to patients treated similarly (e.g., with standard chemotherapy or without specific therapy) but without the first and second agents. The median progression-free survival or overall survival time may also be increased by at least 10 days, but preferably 30 days, 60 days, or 3, 4, 5 or 6 months or 1 year or longer by treatment according to the method of the invention. In addition or alternatively, treatment by the method of the invention may increase the complete response rate, partial response rate, or objective response rate (complete+partial) of patients by at least 10%, 20%, 30% or 40% but preferably 50%, 60% to 70% or even 80%, 90% or 100%. Moreover, when administering treatment with two agents, the regimes with which the respective agents are administered are combined in such a manner that each agent can make a contribution to the therapy, so treatment according to the invention with the first and second agents can increase progression-free or overall survival or increase the complete, partial or objective response rate by at least 10%, 20%, 30% or 40% but preferably 50%, 60% to 70% or even 80%, 90% or 100% compared to treatment with either agent without the other. Indeed, preferably treatment with the first and second agents is synergistic, i.e., better than additive. Optionally, treatment according to the method of the invention can inhibit tumor invasion, or metastasis.

Typically, in a clinical trial (e.g., a phase II, phase II/III or phase III trial), the aforementioned increases in median progression-free survival and/or response rate of the patients treated by the method of the invention together with a standard therapy (e.g., a chemotherapeutic regimen), relative to the control group of patients receiving the standard therapy alone, is statistically significant, for example at the p<0.05 or 0.01 or even 0.001 level. The complete and partial response rates can be determined by objective criteria commonly used in clinical trials for cancer, e.g., as listed or accepted by the National Cancer Institute and/or Food and Drug Administration.

### EXAMPLES

### 1. L2G7 and an Anti-EGFR mAb in a Xenograft Model

The ability of treatment with a first agent that inhibits the activity of HGF (i.e., an HGF antagonist or cMet antagonist) in combination with a second agent that inhibits a signaling pathway other than the one stimulated by HGF (the HGF/cMet pathway) to inhibit human tumor growth is demonstrated in xenograft models in immunodeficient mice or other rodents such as rat. Illustrative but not limiting examples of immunodeficient strains of mice that can be used are nude mice such as CD-1 nude, Nu/Nu, Balb/c nude, NIH-III (NIH-bg-nu-xid BR); scid mice such as Fox Chase SCID (C.B-17 SCID), Fox Chase outbred SCID and SCID Beige; mice deficient in RAG enzyme; as well as nude rats. Experiments are carried out as described previously (Kim et al., Nature 362:841, 1992, which is incorporated herein by reference). Human tumor cells typically grown in complete DMEM medium are typically harvested in HBSS. Female immunodeficient, e.g., athymic nude mice (4-6 wks old) are injected s.c. with typically 5×10⁶ cells in 0.2 ml of HBSS in the dorsal areas. When the tumor size reaches 50-100 mm³, the mice are grouped randomly and appropriate amounts of the agents are administered. For example, an anti-HGF or other mAb (typically between 0.1 and 1.0 mg, e.g. 0.5 mg) is administered i.p. once, twice or three times per week in a volume of, e.g., 0.1 ml, for e.g., 1, 2, 3, or 4 weeks or the duration of the experiment. An orally active small molecule agent may be administered in drinking water or by injection. Tumor sizes are determined typically twice a week by measuring in two dimensions [length (a) and width (b)]. Tumor volume is calculated according to V=ab²/2 and expressed as mean tumor volume±SEM. The number of mice in each treatment group is at least 3, but more often between 5 and 10, e.g., 7. One group of mice is treated with both agents; other groups may be treated with neither agent or with one agent but not the other agent. Omitted agents may optionally be substituted by a “placebo” of like kind, e.g., an irrelevant mAb instead of an active mAb. Statistical analysis may be performed using, e.g., Student's t test. In a variation of this experiment, administration of the agents begins simultaneously or shortly after injection of the tumor cells. The effect of the agents may measured by growth of the tumor with time, prolongation of the survival of the mice, or increase in percent of the mice surviving at a given time or indefinitely.

Various tumor cell lines known to secrete or respond to HGF are used in separate experiments, for example U87 or U118 human glioblastoma cells, and/or GB-d1 human gallbladder tumor cells. Preferred antibodies to be used as the first agent in the invention, such as human-like and reduced-immunogenicity antibodies and the L2G7 antibody and its chimeric and humanized fauns and antibodies with the same epitope as L2G7, when used in combination with the second agent, inhibit growth of tumors by at least 25%, but possibly 40% or 50%, and as much as 75% or 90% or greater, or even completely inhibit tumor growth after some period of time or cause tumor regression or disappearance. There may also be this extent of increased inhibition when both agents are used compared to only one. This inhibition takes place for at least tumor cell lines such as U87 or U118 in at least one mouse strain such as NIH III Beige/Nude, but preferably occurs for 2, 3, several, many, or even essentially all HGF-expressing tumor cell lines of a particular (e.g., glioma) or any type, when tested in one or more immunodeficient mouse strains that do not generate a neutralizing antibody response against the injected antibody. Treatment with some combinations of first and second agents in one or more of the xenograft models leads to the indefinite survival of 50%, 75%, 90% or even essentially all mice, who would otherwise die or need to be sacrificed because of growth of their tumor.

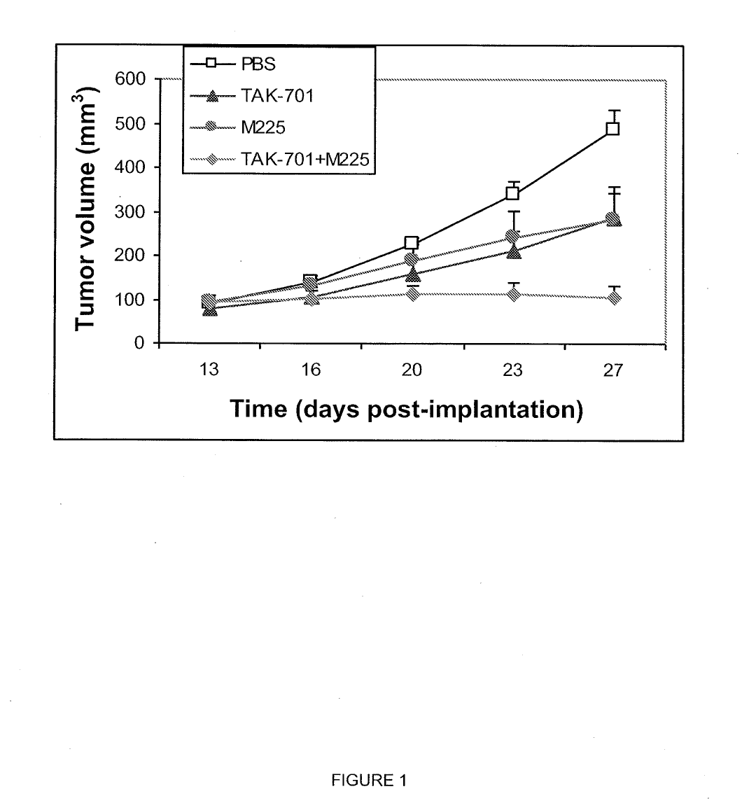

For example, such an experiment was performed with GB-d1 gallbladder tumor xenografts. Female NIH III xid/Beige/nude mice (4-6 wks old) were implanted with tumors by s.c. injection of 10⁶ GB-d1 cells in the dorsal areas. When the tumor size reached ˜100 mm³, the mice were grouped randomly into 4 groups of 5 mice each. Mice in the respective groups received either PBS; humanized L2G7 anti-HGF mAb (also known as HuL2G7 or TAK-701); M225 (the mouse anti-EGFR mAb from which the chimeric cetuximab mAb was derived) or a combination (i.e., both) of HuL2G7 and M225. The mAbs were administered twice per week at 100 μg (approx. 5 mg/kg body weight) from day 13. Tumor sizes were determined twice per week as described above. FIG. 1 shows that while treatment with either HuL2G7 or M225 partially inhibited tumor growth, the combination of mAbs synergistically and completely inhibited tumor growth.

Similar tumor inhibition experiments are performed with the anti-HGF and anti-EGFR mAbs administered together with one or more chemotherapeutic agents (see supra) to which the tumor type is expected to be responsive, as described by Ashkenize et al., J. Clin. Invest. 104:155, 1999. The combination of the two mAbs and chemotherapeutic drug may produce a greater inhibition of tumor growth than either agent alone. The effect may be additive or synergistic, and strongly inhibit growth, e.g. by 80% or 90% or more, or even cause tumor regression or disappearance. The anti-HGF and anti-EGFR mAbs may also be administered in combination with an antibody against another growth or angiogenic factor, for example anti-VEGF, to obtain additive or synergistic growth inhibition and/or tumor regression or disappearance.

### 2. L2G7 and Erlotinib in a Xenograft Model

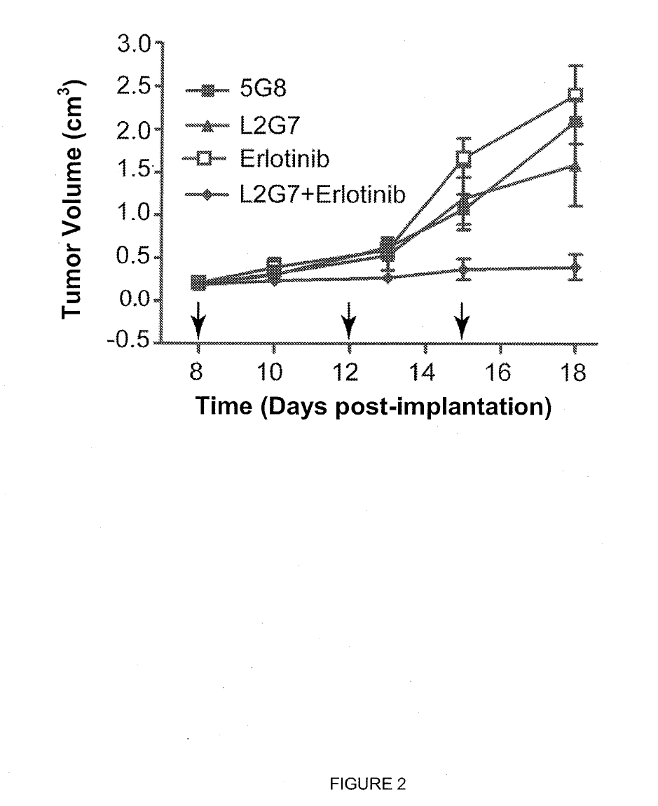

Another experiment utilized a cell line U87EGFRvIII, in which variant III of the EGFR (Ji et al., op. cit.) had been permanently transfected into the U87 glioma cell line. This cell line is a model for the many glioma tumors that express EGFRvIII. The U87EGFRvIII cells were implanted s.c. into immunodeficient mice, which were divided into 4 groups. When the tumors reached approximately 200 mm³ in size, the groups of mice were treated with an irrelevant control mAb 5G8, or with the anti-HGF mAb L2G7, or with the EGFR antagonist erlotinib, or with both L2G7 and erlotinib. The mAbs were delivered i.p. at 5 mg/kg on days 8, 12 and 15, whereas erlotinib (150 mg/kg) was administered 6 times per week. As shown in FIG. 2, L2G7 only inhibited growth of the U87EGFRvIII xenografts modestly, in contrast with its complete inhibition of ordinary U87 xenografts seen in previous experiments. This was due to the increased aggressiveness of the cells induced by the activated EGFRvIII receptor. Likewise, erlotinib only modestly inhibited growth of the tumors. In contrast, treatment with the combination of L2G7 and erlotinib synergistically and almost completely inhibited growth of the U87EGFR xenografts (FIG. 2).

### 3. L2G7 and Erlotinib in an Intracranial Xenograft Model

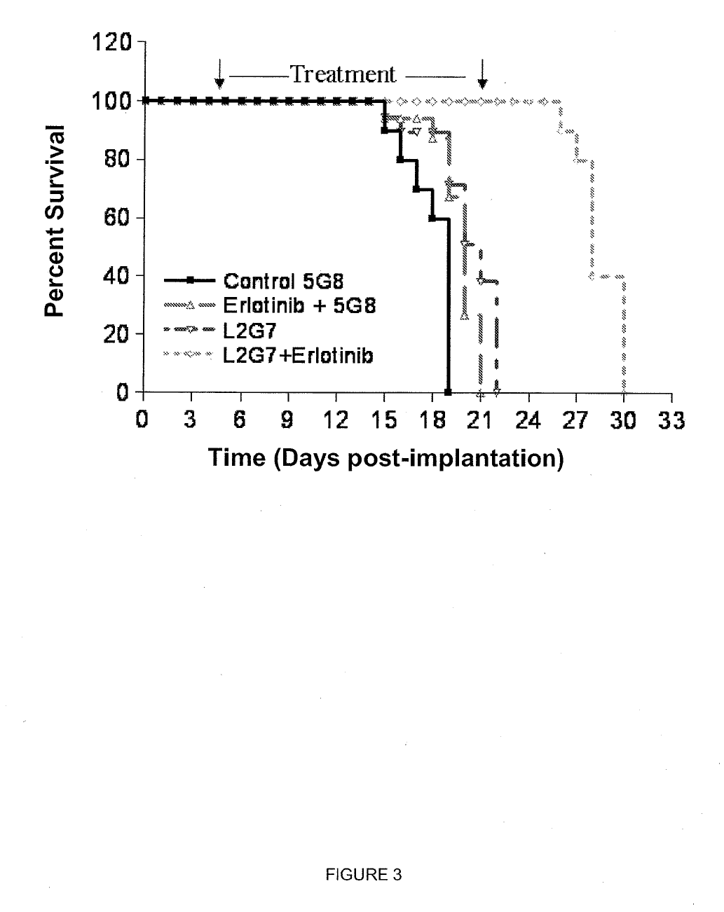

In this experiment, mice were implanted intracranially with U87EGFRvIII cells as described (Kim et al, op. cit.) in order to more accurately simulate brain tumors. Four groups of mice were treated with control mAb 5G8 or anti-HGF mAb L27, either alone or in combination with erlotinib, from post-implantation day 5 to 21 (5 mg/kg mAb twice per week; 150 mg erlotinib 6 times per week). As seen in FIG. 3, treatment with either L2G7 or erlotinib as the only active agent slightly but significantly prolonged survival of the mice relative to treatment with no active agent (5G8 alone): p=0.0012 for erlotinib vs 5G8 and p=0.0004 for L2G7 vs 5G8. In contrast, treatment with the combination of L2G7 and erlotinib prolonged survival much longer than L2G7 or erlotinib alone (p<0.0001 vs any of the other groups). In fact, since treatment was stopped on day 21, it is possible that continued treatment with L2G7 together with erlotinib would have prolonged survival even further. This result and the result in Example 2 show that tumors expressing EGFRvIII, for example gliomas expressing EGFRvIII, are especially suitable for treatment according to the methods of the present invention.

### 4. Sequences of Preferred Anti-HGF mAbs for Use in the Invention

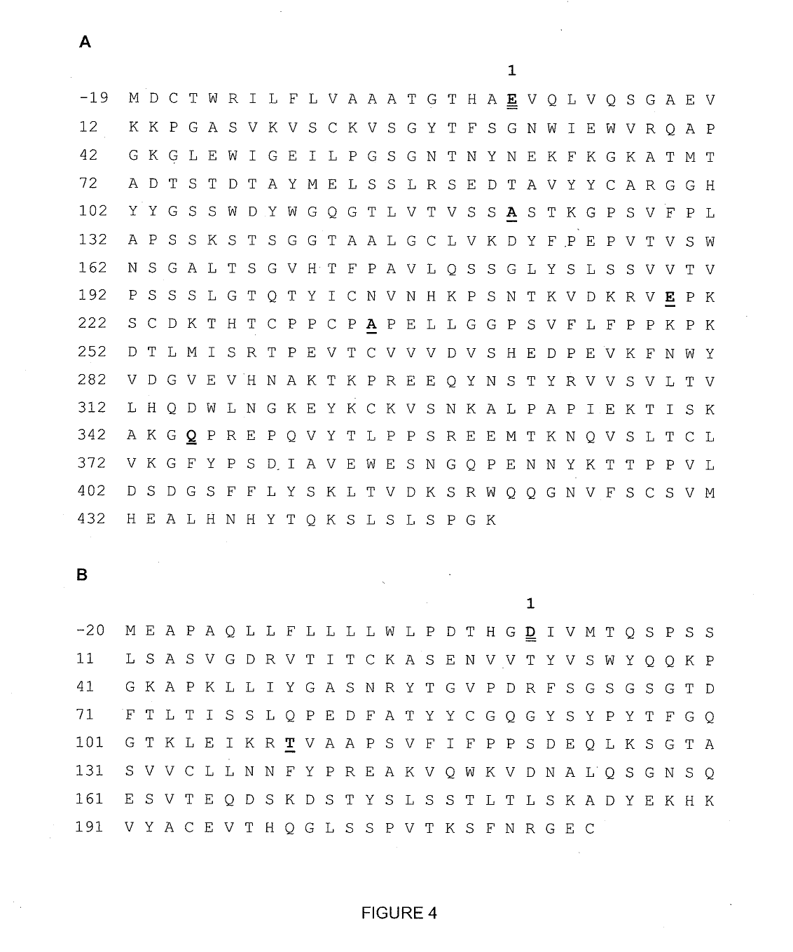

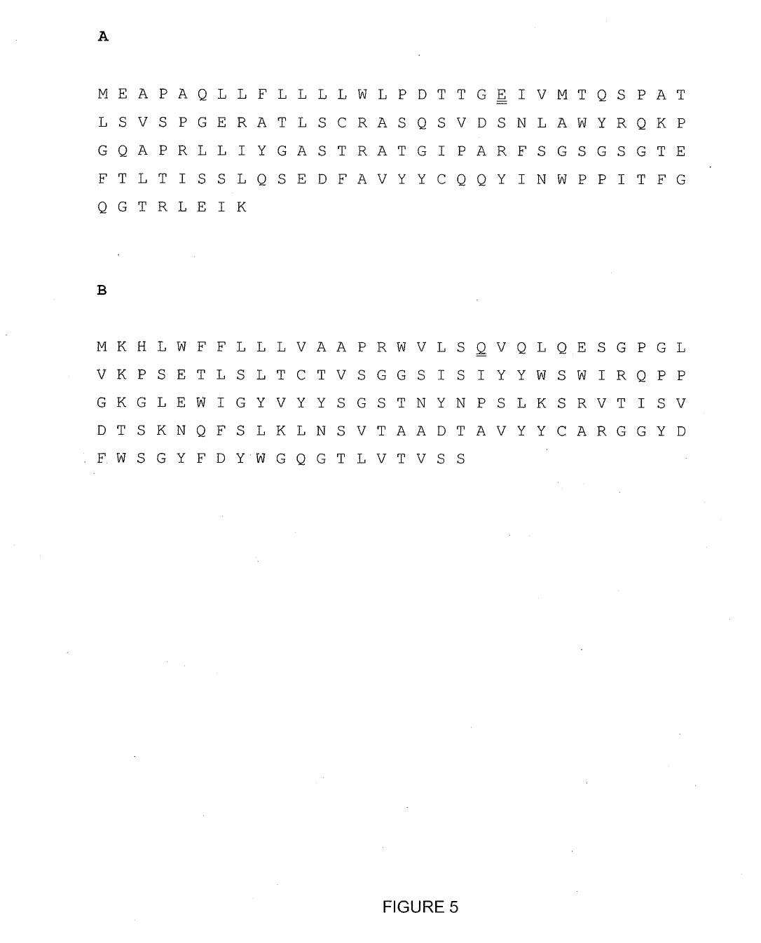

As mentioned above, a humanized form of the neutralizing anti-HGF mAb L2G7, e.g., HuL2G7, is especially preferred as the first agent in the invention. The sequences of the heavy and light chains of HuL2G7 are shown in FIGS. 4A and B, with the first amino acid of the mature sequences (i.e., the first amino acids of the actual mAb HuL2G7) double underlined. The signal sequences preceding the first amino acid of the heavy and light chains of HuL2G7 are cleaved during expression and secretion. The C-terminal lysine of the heavy chain may be cleaved during expression and processing and may not be present in the final product. Also especially preferred for use as the first agent is the anti-HGF mAb 2.12.1 described in WO 2005/017107 A2; the sequences of the variable regions of the light and heavy chains of this mAb are shown in FIGS. 5A and B with the first amino acid of the mature sequences (i.e., the first amino acids of the actual mAb 2.12.1) double underlined. The signal sequences preceding the first amino acid of the heavy and light chains of mAb2.12.1 are cleaved during expression and secretion. The 2.12.1 mAb has as human constant regions adjoined to these light and heavy chain variable region sequences the human kappa constant region and the human gamma-2 constant region respectively, but mAbs with these variable regions and other human constant regions such as gamma-1 are also preferred for use in the invention. MAbs having light and heavy chain variable regions with the same CDRs as those shown in FIGS. 4A and B or FIGS. 5A and 5B are also preferred for use in the invention. MAbs that have amino acid sequences 90%, 95% or 99% identical to those shown in FIGS. 4A and B or FIGS. 5A and B, at least in the CDRs, when aligned according to the Kabat numbering convention, or which differ from FIGS. 4A and B or FIGS. 5A and B by a small number of functionally inconsequential amino acid substitutions (e.g., conservative substitutions), deletions, or insertions, can also be used in the invention, provided they maintain the functional properties of HuL2G7 or 2.12.1 respectively.

Although the invention has been described with reference to the presently preferred embodiments, it should be understood that various modifications can be made without departing from the invention. Unless otherwise apparent from the context any step, element, embodiment, feature or aspect of the invention can be used with any other.

All publications (including GenBank Accession numbers, UniProtKB/Swiss-Prot accession numbers and the like), patents and patent applications cited are herein incorporated by reference in their entirety for all purposes to the same extent as if each individual publication, patent and patent application was specifically and individually indicated to be incorporated by reference in its entirety for all purposes. In the event of any variance in sequences associated with Genbank and UniProtKB/Swiss-Prot accession numbers and the like, the application refers to the sequences associated with the cited accession numbers as of the priority date of the application (Apr. 11, 2008).

U.S. Application Nos. 61/044,444 and 61/044,446 filed Apr. 11, 2008 and PCT applications attorney dockets 022382-000610PC and 022382-000710PC filed on the same day as the present application are also directed to methods of treating cancer by combination of inhibitors of HGF and a second agent inhibiting a second pathway. Unless otherwise apparent from the context, any step, element, embodiment, feature or aspect of the present application can be combined with any step, element, embodiment, feature or aspect of U.S. Application Nos. 61/044,444 and 61/044,446, or PCT applications 022382-000610PC and 022382-000710PC, all of which are incorporated by reference.

ATCC Number PTA-5162 has been deposited at the American Type Culture Collection, P.O. Box 1549 Manassas, Va. 20108, as ATCC Number PTA-5162 under the Budapest Treaty. This deposit will be maintained at an authorized depository and replaced in the event of mutation, nonviability or destruction for a period of at least five years after the most recent request for release of a sample was received by the depository, for a period of at least thirty years after the date of the deposit, or during the enforceable life of the related patent, whichever period is longest. All restrictions on the availability to the public of these cell lines will be irrevocably removed upon the issuance of a patent from the application.

We claim:

1. A method of treating cancer in a patient comprising administering to the patient a first agent that is an inhibitor of Hepatocyte Growth Factor (HGF) in combination with a second agent that is an inhibitor of a cellular signaling pathway other than the HGF/cMet pathway.

2. The method of claim 1 wherein said first agent is a monoclonal antibody.

3. The method of claim 2 wherein the monoclonal antibody binds to and neutralizes HGF as a single agent.

4. The method of claim 3 wherein the monoclonal antibody is human, humanized or chimeric.

5. The method of claim 4 wherein the monoclonal antibody is humanized.

6. The method of claim 5 wherein the monoclonal antibody is a humanized L2G7 antibody.

7. The method of claim 4 wherein the monoclonal antibody is human.

8. A method of treating cancer in a patient comprising administering to the patient an inhibitor of Hepatocyte Growth Factor (HGF) in combination with an inhibitor of Epidermal Growth Factor (EGF).

9. The method of claim 8 wherein the inhibitor of HGF is a monoclonal antibody.

10. The method of claim 9 wherein the monoclonal antibody binds to and neutralizes HGF as a single agent.

11. The method of claim 10 wherein the monoclonal antibody is genetically engineered.

12. The method of claim 10 wherein the monoclonal antibody is human.

13. The method of claim 10 wherein the monoclonal antibody is humanized.

14. The method of claim 13 wherein the monoclonal antibody is a humanized L2G7 antibody.

15. The method of claim 8 wherein the inhibitor of EGF is an EGF receptor (EGFR) antagonist.

16. The method of claim 15 wherein the EGFR antagonist is a monoclonal antibody that binds EGFR, thereby inhibiting binding of EGF to EGFR.

17. The method of claim 16 wherein the monoclonal antibody is cetuximab or panitumumab.

18. The method of claim 15 wherein the EGFR antagonist is erlotinib or gefitinib.

19. The method of claim 8 wherein the cancer is selected from the group of lung cancer, colon cancer, and head and neck cancer.

20. The method of claim 8 wherein the cancer is glioma.

21. The method of any preceding claim, wherein the cancer includes cells with a mutant EGFR gene.

22. The method of claim 21, wherein the mutation is a deletion of exons 2-7 of the EGFR gene.

23. A composition or kit comprising a humanized L2G7 antibody and drug selected from the group consisting of cetuximab, panitumumab, erlotinib and gefitib.


SEQUENCE LISTING (8 sequences)

SEQ ID NO: 1 - protein, 469 aa, Artificial Sequence
  Description of Artificial Sequence Synthetic polypeptide
MDCTWRILFLVAAATGTHAEVQLVQSGAEVKKPGASVKVSCKVSGYTFSGNWIEWVRQAPGKGLEWIGEILPGSGNTNYNEKFKGKATMTADTSTDTAYMELSSLRSEDTAVYYCARGGHYYGSSWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 234 aa, Artificial Sequence
  Description of Artificial Sequence Synthetic polypeptide
MEAPAQLLFLLLLWLPDTHGDIVMTQSPSSLSASVGDRVTITCKASENVVTYVSWYQQKPGKAPKLLIYGASNRYTGVPDRFSGSGSGTDFTLTISSLQPEDFATYYCGQGYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 128 aa, Artificial Sequence
  Description of Artificial Sequence Synthetic polypeptide
MEAPAQLLFLLLLWLPDTTGEIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIK

SEQ ID NO: 4 - protein, 139 aa, Artificial Sequence
  Description of Artificial Sequence Synthetic polypeptide
MKHLWFFLLLVAAPRWVLSQVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSS

SEQ ID NO: 5 - protein, 214 aa, Artificial Sequence
  Description of Artificial Sequence Synthetic polypeptide
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 445 aa, Artificial Sequence
  Description of Artificial Sequence Synthetic polypeptide
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 127 aa, Artificial Sequence
  Description of Artificial Sequence Synthetic polypeptide
MRAPAQFLGFLLFWIPASRSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 8 - protein, 138 aa, Artificial Sequence
  Description of Artificial Sequence Synthetic polypeptide
MAVLALLFCLVTFPSCVLSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA